(19) United States
(12) Patent Application Publication
(10) Pub. No.: US 2018/0345028 A1
(43) Pub. Date: Dec. 6, 2018

(54) IMPLANTABLE DEFIBRILLATOR HEART PUMP WITH INTEGRATED DEFIBRILLATOR COMPONENTS

(71) Applicants: Anas Aboud, Rinteln (DE); Kai Liebing, Gera (DE)

(72) Inventors: Anas Aboud, Rinteln (DE); Kai Liebing, Gera (DE)

(21) Appl. No.: 15/939,379

(22) Filed: Mar. 29, 2018

(30) Foreign Application Priority Data
Mar. 29, 2017 (DE) 20 2017 001 760

(51) Int. Cl. A61N 1/39; A61M 1/12; A61N 1/378; A61N 1/05; A61B 17/34

(52) U.S. Cl. CPC A61N 1/3962; A61M 1/122; A61M 1/127; A61M 2210/125; A61N 1/0595; A61B 17/3468; A61M 2205/054; A61N 1/3787

(57) ABSTRACT

An implantable defibrillator heart pump includes an integrated replaceable difibrillator device. The defibrillator heart pump includes a pump head, a percutaneous line/combined drive line, a pair of implanted cardioverter defibrillator electrodes and an outflow prosthesis.

### FIELD OF INVENTION

The disclosed technology relates generally to ventricular assist devices (VADs), and, more particularly, to a defibrillator heart pump having an artificial heart pump system and integrated defibrillator components.

### BACKGROUND OF THE INVENTION

VADs are artificial, surgically-implanted heart pumps used primarily in patients with severe, end stage heart failure to assist or relieve ventricular function. The VAD systems are used in acute as well as chronic heart failure. The goal is to improve the pumping power of the heart. In some patients, the VAD is a bridge to transplant, while in others, it is considered temporary and may be removed following improved ventricular function, for example, as in the case of acute myocarditis. However, in most patients, these devices are considered the definitive therapy.

### SUMMARY OF THE INVENTION

Aspects of the disclosed technology relate to a defibrillator heart pump in the form of an artificial heart pump system integrated with a replaceable defibrillator device.

One aspect of the disclosed technology relates to a defibrillator heart pump that includes a pump head; a percutaneous/combined drive line; a pair of electrodes operatively coupled to the percutaneous/combined drive line; and an outflow prosthesis operatively coupled to the pump head.

According to one feature, the pair of electrodes are shock electrodes that are configured to be externally inserted, removed and/or replaced via a guide tube running through the percutaneous line. According to one feature, the electrodes are configured to be coupled to and/or disconnected from an associated ventricular assist device without stopping operation of the pump head.

According to one feature, the electrodes are configured to be powered by a battery and/or a power supply associated with the pump head.

According to one feature, the defibrillator heart pump is configured to provide automatic shock delivery via the pair of electrodes.

According to one feature, the defibrillator heart pump is configured to provide manual suppression of automatic shock delivery upon external activation.

Another aspect of the disclosed technology relates to a medical device having an artificial heart pump system integrated with a replaceable defibrillator device, the medical device that includes a pump head; a percutaneous/combined drive line; a pair of electrodes operatively coupled to the percutaneous/combined drive line; and an outflow prosthesis operatively coupled to the pump head.

According to one feature, the medical device includes a ventricular assist device (VAD) controller operatively coupled to the artificial heart pump system and an implanted cardioverter defibrillator (ICD) controller operatively coupled to the replaceable defibrillator device.

According to one feature, the pair of electrodes are shock electrodes that are configured to be externally inserted, removed and/or replaced via a guide tube running through the percutaneous line.

These and further features of the disclosed technology will be apparent with reference to the following description and attached drawings. In the description and drawings, particular embodiments or aspects of the disclosed technology have been disclosed in detail as being indicative of some of the ways in which the principles of the disclosed technology may be employed, but it is understood that the disclosed technology is not limited correspondingly in scope. Rather, the disclosed technology includes all changes, modifications and equivalents coming within the spirit and terms of the claims appended thereto.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

### BRIEF DESCRIPTION OF THE DRAWINGS

These and other features of the disclosed technology, and their advantages, are illustrated specifically in embodiments of the disclosed technology now to be described, by way of example, with reference to the accompanying diagrammatic drawings, in which:

FIG. 1 is a diagrammatic illustration of a defibrillator heart pump in accordance with one aspect of the disclosed technology.

It should be noted that all the drawings are diagrammatic and not drawn to scale. Relative dimensions and proportions of parts of these figures have been shown exaggerated or reduced in size for the sake of clarity and convenience in the drawings. The same reference numbers are generally used to refer to corresponding or similar features in the different embodiments. Accordingly, the drawing(s) and description are to be regarded as illustrative in nature and not as restrictive.

### DETAILED DESCRIPTION OF THE INVENTION

As is discussed more fully below, aspects of the disclosed technology relate to a defibrillator heart pump in the form of an artificial heart pump system integrated with a replaceable defibrillator device. In accordance with one exemplary embodiment, the defibrillator heart pump can include a pump head, percutaneous line/combined driveline electrodes (e.g., implantable cardioverter defibrillator (ICD) electrodes), and an outflow prosthesis. Via the percutaneous line of this defibrillator heart pump system, the patient's diseased heart can be defibrillated or stimulated either temporarily or permanently. Shock is also possible during pump activity. The defibrillator elements can be easily replaced in case of a defect without disturbance of pump function. As discussed more fully below, the defibrillator heart pump can include an artificial heart pump system, (also referred to as a ventricular assist device (VAD)) and integrated defibrillator components. The intracorporeal heart pump is preferably located in the apex of the left ventricle, (e.g., at the tip of the diseased heart), but can be applied to other locations without departing from the scope of the disclosed technology.

VADs are artificial, surgically-implanted heart pumps used primarily in patients with severe, end stage heart failure to assist or relieve ventricular function. The VAD systems are used in acute as well as chronic heart failure. The goal is to improve the pumping power of the heart. In some patients, the VAD is a bridge to transplant, while in others, it is considered temporary and may be removed following improved ventricular function, for example, as in the case of acute myocarditis. However, in most patients, these devices are considered the definitive therapy.

The main complications of VADs are thrombembolic events, bleeding, infection (e.g., driveline and surgical site infection), and right heart failure in the early phase after implantation. Malignant cardiac arrhythmias such as ventricular tachycardia or ventricular fibrillation are more common in patients with VAD systems. It is proposed that these arrhythmias are likely due to existing damage to the heart muscle. The VAD provides stability of circulation despite arrhythmias due to constant flow generated by the device. Nevertheless, arrhythmias affecting the right ventricle may result in decompensated right heart failure, which in turn may cause other organ dysfunction such as renal insufficiency. Bedi et al. demonstrated the cumulative higher mortality in VAD patients with ventricular arrhythmias.

Ventricular arrhythmias, such as ventricular flutter and ventricular fibrillation, may be treated with an automatic implanted cardioverter defibrillator (AICD or ICD). These devices independently detect ventricular arrhythmias and treat them using one or more pre-programmed algorithms according to the underlying arrhythmia detected. This is done by electrical defibrillation. The implantation of an ICD is usually a stand-alone procedure typically done under general anesthesia.

The AICD can be implanted subcutaneously or submuscularly below the left clavicle using an approximately 5 centimeter (cm) incision. The ICD electrodes are introduced either via a superficial vein or via insertion of through the subclavian vein under fluoroscopy. The electrode is then placed at the tip of the right ventricle. The electrodes, which include two parts, are used to monitor the ventricular rhythm and to detect life-threatening arrhythmias. Depending on the model, one or two coils are located within the electrodes, through which the delivery of the electrical shock takes place during a necessary defibrillation. The electrical shock flows either between the ICD and the electrode or between two electrodes. The aim is to defibrillate the largest possible area of myocardium. The electric shock then converts the ventricular arrhythmia to a normal rhythm. It will be appreciated that a device has been proposed to offer mechanical support to the ventricle by local compression and to control the rhythm of the heart.

ICD implantation is a well-established procedure carried out by both cardiologists as well as cardiac surgeons. These operations successfully treat many patients with heart disease. However, like any other surgical procedure, there is the unavoidable risk of postoperative complications. In a review done by Ezzat et al., which studied a large number of ICD patients, the rate of deficiency-related complications with the need for re-intervention or re-hospitalization was 9.1%. AICD complications can be divided into 4 categories:

1. Line-related complications such as venous thrombosis, pneumothorax or hematothorax (following venipuncture), and ICD-pocket hematoma

2. Electrode-related complications such as vein perforation, right ventricle perforation (possibly leading to a cardiac tamponade), probe dislocation, probe break or defect, and dysfunction of the electrodes

3. Aggregate-related complications such as migration of the ICD which as a rule requires operative revision

4. Infection-related complications including surgical site, ICD-pocket, and electrodes site infections. These infections usually require removal of the device

Of note, patients with advanced cardiac disease receiving a VAD have between 22% and 52% risk of ventricular arrhythmias with increased risk of mortality and morbidity. It has been shown that ICD implantation may offer significant reduction in mortality of up to 39% in these patients. ICD implantation, as a stand-alone procedure, is inherently risky and can cause various undesirable complications including infection, bleeding, and dislocation of aggregate or probes. The psychological implications of ICD implantation are also well documented including higher rates of depression and anxiety which may significantly affect quality of life.

Based on the above, in order to avoid these potential complications in VAD patients, aspects of the disclosed technology relate to a defibrillator heart pump system.

The disclosed technology is based on the goal of further optimizing VADs and to increase patient safety. This is achieved by the development of an artificial heart pump system or VAD with an integrated defibrillator function, which would be used as an integrated unit with a switch-on, switch off capability. More VAD patients than initially suspected were found to have received no ICD with the VAD.

In our first in-vitro experiment with a self-built defibrillator ventricular assist device functional model in Jena and Bad Oeynhausen, we were unable to detect any functional impairment in the running heart pump/VAD with several defibrillators of up to 50 J. The use of these prototypes is currently being prepared as part of an in-vivo animal testing at the University Hospital laboratory in Jena.

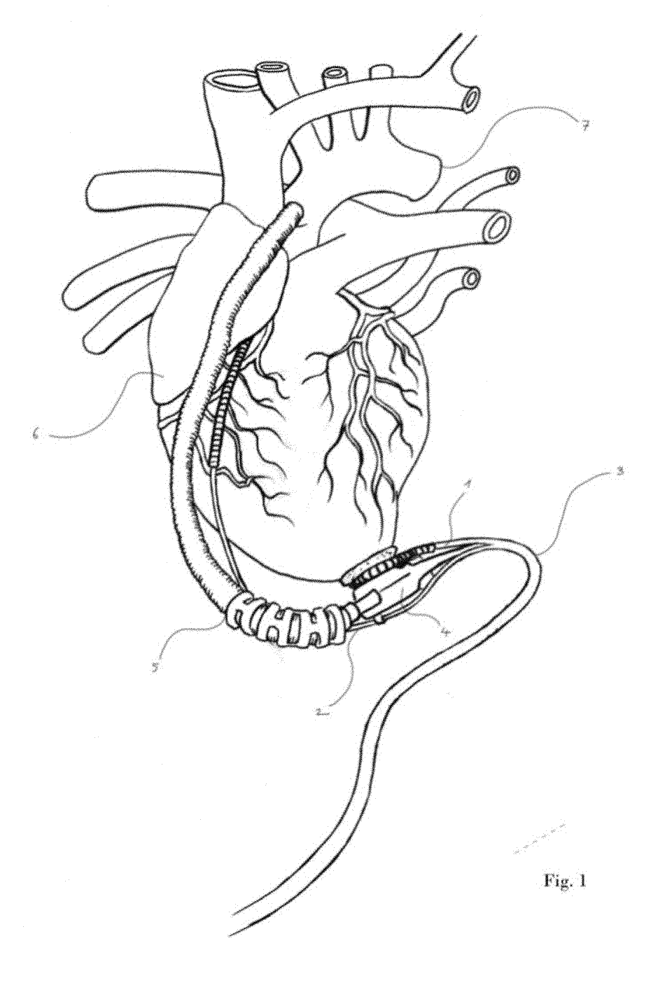

FIG. 1 depicts an exemplary embodiment of a defibrillator heart pump in accordance with the disclosed technology. The defibrillator heart pump supports the diseased heart with the illustrated integrated defibrillator electrodes 1 and 2. It will be appreciated that FIG. 1 shows the implanted part of the defibrillator heart pump and illustrates the disclosed technology with its various components. The defibrillator electrodes are integrated into the heart pump system, which pumps blood from the left ventricle into the aorta (indicated by reference numeral 7). In the illustrated embodiment, one of the electrodes 1 is completely connected to the pump head 4. It will be appreciated that this aspect of the exemplary illustrated embodiment is technically simple, because the electrode and the VAD housing have a similar metal structure. The second electrode 2 runs along the outflow prosthesis 5 to, for example, behind the right atrial appendage (indicated by reference numeral 6). A normal single-coil electrode (with only one shock coil) or a suitable epicardial electrode could be used without departing from the scope of the disclosed technology. The shock delivery takes place via a short path between electrodes 1 and 2.

Electrodes to detect heart rhythm can also be integrated distally or proximally to the shock coil and an additional sensing electrode would not be necessary. Both electrodes 1 and 2 can be extra-corporeally discharged via the percutaneous line (also referred to as the ventricular assist device driveline or the VAD driveline) 3. It will be appreciated that the percutaneous line can also be thought of as a driveline and/or connection between intra-corporeal heart pump and external VAD controller.

The control unit (e.g., controller and battery) of the VAD system with the integrated defibrillator can be connected or otherwise operatively coupled to the above-mentioned electrodes. It will be appreciated that those components (e.g., controller, battery, defibrillator) could be carried in an appropriate belt or an appropriate shoulder-bag. This new technique may avoid the possible risks of ICD implantation such as infection and electrodes and aggregate dislocations. The risk of hematoma, pneumothorax, perforation, or cardiac tamponade would no longer present. Furthermore, replacement of the ICD aggregate with battery depletion by the disclosed technology requires no surgical intervention, and may be carried out extracorporeally or, alternatively, the VAD power supply may also supply the ICD. The ICD can be easily connected to the VAD control unit. Thin guide tubes inside the percutaneous lead 3 allow defibrillatory electrodes 1 and 2 to be externally inserted, removed, or replaced without stopping the heart pump.

The more complex the system, the more susceptible it is to failure, and the higher the risk of a life-threatening pump stop or a malfunction of the heart pump system, can be eliminated. To prevent erroneous shock deliveries, a plausibility assessment between the VAD control unit and external ICD unit could take place before shock delivery. In addition, the conscious patient could also prevent or delay a shock delivery at the push of a button. A professional rescuer would also be able to trigger a shock manually. For life-threatening cardiac arrhythmias (e.g., ventricular fibrillation, ventricular flutter, and ventricular tachycardia), an automatic speed adjustment of the heart pump would also be possible, in addition to the defibrillation. A simple pacemaker function, for example, to treat bradycardia, would also be conceivable. Furthermore, due to the wide area of contact of the first defibrillator pole via the VAD housing, there is a better sensing of the electrical myocardial signal. This could lead to the avoidance or reduction of the faulty shock charges. Due to the epicardial attachment of the probes, tricuspid valve dysfunction caused by endocardial electrodes is preventable. Reliability and effectiveness of this proposed device can allow for an antibradycardiac or antitachycardic stimulation therapy to be explored in the near future with in vivo animal testing. Telemedical connection and continuous sensing control are possible. Thus, the data from the controller and unit could be checked regularly. Other benefits include reducing programmer and VAD interference from the magnetic pump action, as the ICD aggregate is extracorporeal. With this disclosed technology, by avoiding implanting the ICD as a separate procedure, medical costs can be reduced. Also, the cost of building an extracorporeal defibrillator is significantly less than that of an implantable device.

The mandatory, patient-related safety checks of the external components of the heart pump system and the ICD unit may be performed together or separately.

When the technology is more advanced, it is also conceivable that the ICD unit may be fully integrated into the VAD controller and implanted together as one unit into the patient. An implanted battery could then be charged via induction through the skin. However, this would require long term use and testing of the defibrillator-heart. An exchange of the ICD unit or a change from the VAD controller, or implanted battery, for example in case of a defect, would not be easy with a complete implantation. Currently, most VAD patients have a spare controller at their fingertips which may be changed independently and, if needed, urgently by patient or family member. The VAD is often referred to as an artificial heart, although it usually only supports the left ventricle (Left Ventricular Assist Device or LVAD). In extremely rare cases, a total Artificial Heart (TAH), which completely replaces the heart, is used. This is a last resort therapy for patients with complete end stage heart failure and is considered to be a bridge to heart transplantation. The mortality in this group of patients is typically high. The TAH system does not offer patients continuous therapy with a maximum duration of therapy of 903 days. Unlike this system, an LVAD pump can offer a much longer treatment option (most likely between 10 and 20 years). The LVAD system or the defibrillator-heart pump system is less costly than a TAH and in most patients with advanced, especially left-sided cardiomyopathy, it is usually sufficient. In the case of a technical defect, the weak biological heart of the patient acts as a backup giving enough time to address the malfunction successfully.

Although the disclosed technology has been shown and described with respect to a certain preferred aspect, embodiment or embodiments, it is obvious that equivalent alterations and modifications will occur to others skilled in the art upon the reading and understanding of this specification and the annexed drawings. In particular regard to the various functions performed by the above described elements (components, assemblies, devices, members, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary aspect, embodiment or embodiments of the disclosed technology. In addition, while a particular feature of the disclosed technology may have been described above with respect to only one or more of several illustrated aspects or embodiments, such feature may be combined with one or more other features of the other embodiments, as may be desired and advantageous for any given or particular application.

1. A defibrillator heart pump comprising:
a pump head;
a percutaneous/combined drive line;
a pair of electrodes operatively coupled to the percutaneous/combined drive line; and
an outflow prosthesis operatively coupled to the pump head.

2. The defibrillator heart pump of claim 1, wherein the pair of electrodes are shock electrodes that are configured to be externally inserted, removed and/or replaced via a guide tube running through the percutaneous line.

3. The defibrillator heart pump of claim 2, wherein the electrodes are configured to be coupled to and/or disconnected from an associated ventricular assist device without stopping operation of the pump head.

4. The defibrillator heart pump of claim 2, wherein the electrodes are configured to be powered by a battery and/or a power supply associated with the pump head.

5. The defibrillator heart pump of claim 2, wherein the defibrillator heart pump is configured to provide automatic shock delivery via the pair of electrodes.

6. The defibrillator heart pump of claim 5, wherein the defibrillator heart pump is configured to provide manual suppression of automatic shock delivery upon external activation.

7. A medical device having an artificial heart pump system integrated with a replaceable defibrillator device, the medical device comprising:
a pump head;
a percutaneous/combined drive line;
a pair of electrodes operatively coupled to the percutaneous/combined drive line; and
an outflow prosthesis operatively coupled to the pump head.

8. The medical device of claim 7, further comprising a ventricular assist device (VAD) controller operatively coupled to the artificial heart pump system and an implanted cardioverter defibrillator (ICD) controller operatively coupled to the replaceable defibrillator device.

9. The medical device of claim 8, wherein the pair of electrodes are shock electrodes that are configured to be externally inserted, removed and/or replaced via a guide tube running through the percutaneous line.

10. The medical device of claim 9, wherein the electrodes are configured to be coupled to and/or disconnected from the medical device without stopping operation of the artificial heart pump system.

11. The defibrillator heart pump of claim 9, wherein the electrodes are configured to be powered by a battery and/or a power supply associated with the VAD controller.

12. The medical device of claim 9, wherein the medical device is configured to provide automatic shock delivery via the pair of electrodes.

13. The medical device of claim 12, wherein the medical device is configured to provide manual suppression of automatic shock delivery upon external activation.

14. The medical device of claim 9, wherein the medical device is configured such that the replaceable defibrillator device can be replaced without disturbance of function of the artificial heart pump.
